(19) United States
(12) Patent Application Publication
(10) Pub. No.: US 2021/0085676 A1
(43) Pub. Date: Mar. 25, 2021

(54) TREATMENT FOR AGE- AND OXIDATIVE STRESS-ASSOCIATED MUSCLE ATROPHY AND WEAKNESS

(71) Applicants: Oklahoma Medical Research Foundation, Oklahoma City, OK (US); Department of Veterans Affairs, Washington, DC (US)

(72) Inventors: Holly Van Remmen, Arcadia, OK (US); Rizwan Qaisar, Oklahoma City, OK (US)

(21) Appl. No.: 16/970,607

(22) PCT Filed: Jan. 15, 2019
(86) PCT No.: PCT/US2019/013644

§ 371 Date: Aug. 17, 2020

Related U.S. Application Data
(60) Provisional application No. 62/633,958, filed on Feb. 22, 2018.

(51) Int. Cl. A61K 31/496; A61K 31/15; G01N 33/68; A61K 31/05; A61K 31/47; A61P 21/00

(52) U.S. Cl. CPC A61K 31/496; A61K 31/15; A61P 21/00; A61K 31/05; A61K 31/47; G01N 33/6887

(57) ABSTRACT

The present invention includes methods and compositions for treating a skeletal muscular atrophy caused by a defect in the function of one or more sarco/endoplasmic reticulum Ca²⁺-ATPase (SERCA) pumps comprising: identifying a subject having a muscular atrophy caused by a defect in the function of the one or more SERCA pumps, and providing the subject with an effective amount of an activator that enhances an activity of the one or more SERCA pumps.

### TECHNICAL FIELD OF THE INVENTION

The present invention relates in general to the field of muscular atrophy (more specifically sarcopenia), muscular degeneration, muscular dystrophy (more specifically Duchenne muscular dystrophy), denervation, reduced cardiac function and age-related muscle degeneration, muscular atrophy and weakness caused by one or more defects in Sarcoplasmic/Endoplasmic Reticulum Ca²⁺-ATPase (SERCA) activity.

### BACKGROUND OF THE INVENTION

Without limiting the scope of the invention, its background is described in connection with sarcopenia.

Sarcopenia is a condition that is characterized by loss of muscle mass, muscle strength and muscle functional impairment with ageing. Sarcopenia can be precipitated by a number of factors, including age, nutritional deficiencies, hormonal changes, metabolic disturbance, comorbidities, inflammation, drug adverse effects, genetic predisposition and the effect of the environment. This results in reduction in muscle mass and strength, leading to sarcopenic status, which in turn leads to weakness and a reduced mobility, with downstream deconditioning and reduced physiological reserve. The muscle weakness and reduced mobility in sarcopenia leads to a propensity for reduced physical exercise and activity, which leads to further wasting of muscle and loss of muscle strength, and thus completing a downward spiral into sarcopenia.

Sarcopenia is a major component of decreased health span in the elderly. Although the underlying mechanisms of sarcopenia are still not well defined, intracellular calcium dysregulation has been identified as an important contributor. In support of this, the activity of SERCA, which controls cytosolic calcium levels by returning calcium to the SR following muscle contraction, is reduced in aging skeletal muscle. The primary treatment for sarcopenia is exercise, specifically resistance training or strength training. These activities increase muscle strength and endurance using weights or resistance bands. Although drug therapy is not the preferred treatment for sarcopenia, a few medications are under investigation. These drug therapies include, anabolic or androgenic steroids, selective androgen receptor modulators, protein anabolic agents, appetite stimulants, myostatin inhibitors, activin II receptor drugs, β receptor blockers, ACE inhibitors and troponin activators. Collectively, these treatments offer varying degrees of efficacy. However, these drug therapies also cause a number of more serious side effects, such as, adverse cardiovascular effects, telangiectasia, epistaxis and deranged gonadotropin levels.

Previous studies have used genetic approaches to stabilizing SERCA activity with elevated levels of Hsp72 (20), deletion of the SERCA inhibitor sarcolipin (11) or overexpression of SERCA protein itself (15), which ameliorated muscle pathology in mouse models of Duchenne muscular dystrophy.

One example of such a therapy is taught in U.S. Pat. No. 6,670,386, issued to Sun, et al., and entitled “Bicyclic Modulators of Androgen Receptor Function”, which is said to teach methods for using adherent placental stem cells and placental stem cell populations, and methods of culturing, proliferating and expanding the same, and methods of differentiating the placental stem cells. These inventors are also said to teach methods of using bicyclic compounds in the treatment of androgen receptor-associated age related diseases such as sarcopenia, and to pharmaceutical compositions containing such compounds.

Another example is found in U.S. Pat. No. 8,063,188, filed by Sayers, et al., entitled “Anti-Myostatin Antibodies”. The invention embodies the use of an antibody of the invention for the preparation of a medicament for the treatment of muscle wasting, frailty, age-related sarcopenia, disuse atrophy and cachexia.

However, to-date there is still a major requirement for an effective, safe small molecule treatment for muscular atrophy (more specifically sarcopenia), muscular degeneration, muscular dystrophy (e.g., Duchenne muscular dystrophy), denervation, reduced cardiac function and age-related muscle degeneration, muscular atrophy and weakness caused by one or more defects in Sarcoplasmic/Endoplasmic Reticulum Ca²⁺-ATPase (SERCA) activity.

### SUMMARY OF THE INVENTION

Sarcopenia or age-related muscle atrophy and weakness is associated with a number of pathophysiologic processes such as increased oxidative stress and disruption of intracellular calcium homeostasis. Currently there are no effective pharmacological treatments to reduce the impact of sarcopenia because the mechanism is poorly understood. Herein the inventors show that targeting improved calcium homeostasis through pharmacological restoration of SERCA activity using the compound CDN1163, an allosteric activator of SERCA, acts to blunt muscle atrophy, restore muscle strength and reduce oxidative stress in Sod1^{-/-} mice. These findings provide a promising approach toward therapeutic intervention of sarcopenia and a better understanding of the underlying mechanisms. In another one aspect, the activator is adapted for oral, intravenous, intramuscular, cutaneous, subcutaneous, rectal, nasally, pulmonary, or transdermal administration.

In one embodiment, the present invention includes a method of treating a skeletal muscular atrophy caused by a defect in the function of one or more sarco/endoplasmic reticulum Ca²⁺-ATPase (SERCA) pumps comprising: identifying a subject having a muscular atrophy caused by a defect in the function of the one or more SERCA pumps; and providing the subject with an effective amount of an activator that enhances an activity of the one or more SERCA pumps. In one aspect, the skeletal muscular atrophy is age-related. In another aspect, the skeletal muscular atrophy is oxidative stress-related. In another aspect, the skeletal muscular atrophy is sarcopenia. In another aspect, the SERCA pump is selected from SERCA1, 2, 3 or any isoforms of SERCA1, 2 or 3. In another aspect, the activator is selected from CDN1163, ranolazine, istaroxime, or gingerol. In another one aspect, the activator is adapted for oral, intravenous, intramuscular, cutaneous, subcutaneous, rectal, nasally, pulmonary, or transdermal administration.

In another embodiment, the present invention includes a method of treating a skeletal muscular degeneration caused by a defect in the function of one or more sarco/endoplasmic reticulum Ca²⁺-ATPase (SERCA) pumps comprising: identifying a subject having a muscular degeneration caused by a defect in the one or more SERCA pumps; and providing the subject with an effective amount of an activator that enhances an activity of the one or more SERCA pumps. In one aspect, the skeletal muscular degeneration is age-related. In another aspect, the skeletal muscular degeneration is oxidative stress-related. In another aspect, the skeletal muscular degeneration causes muscular atrophy. In another aspect, the muscular atrophy is age-related. In another aspect, the muscular atrophy is oxidative stress-related. In another aspect, the SERCA pump is selected from SERCA1, 2, 3 or any isoforms of SERCA1, 2 or 3. In another aspect, the activator is selected from CDN1163, ranolazine, istaroxime, or gingerol. In another one aspect, the activator is adapted for oral, intravenous, intramuscular, cutaneous, subcutaneous, rectal, nasally, pulmonary, or transdermal administration.

In another embodiment, the present invention includes a method of treating muscular dystrophy caused by a defect in the function of one or more sarco/endoplasmic reticulum Ca²⁺-ATPase (SERCA) pumps comprising: identifying a subject having a denervation caused by a defect in the function of the one or more SERCA pumps; and providing the subject with an effective amount of an activator that enhances an activity of the one or more SERCA pumps. In one aspect, the muscular dystrophy is age-related. In another aspect, the muscular dystrophy is oxidative stress-related. In another aspect, the muscular dystrophy causes muscular atrophy. In another aspect, the muscular atrophy is age-related. In another aspect, the muscular atrophy is oxidative stress-related. In another aspect, the muscular dystrophy is Duchenne muscular dystrophy. In another aspect, the SERCA pumps is/are selected from SERCA1, 2, 3 or any isoforms of SERCA1, 2 or 3. In another aspect, the activator is selected from CDN1163, ranolazine, istaroxime, or gingerol. In another one aspect, the activator is adapted for oral, intravenous, intramuscular, cutaneous, subcutaneous, rectal, nasally, pulmonary, or transdermal.

In another embodiment, the present invention includes a method of treating denervation caused by a defect in the function of one or more sarco/endoplasmic reticulum Ca²⁺-ATPase (SERCA) pumps comprising: identifying a subject having a denervation caused by a defect in the function of the one or more SERCA pumps; and providing the subject with an effective amount of an activator that enhances an activity of the one or more SERCA pumps. In one aspect, the denervation is symptomatic of hyperthyroidism. In another aspect, the denervation is age-related. In another aspect, the denervation is oxidative stress-related. In another aspect, the one or more SERCA pumps is selected from SERCA1, 2, 3 or any isoforms of SERCA1, 2 or 3. In another aspect, the activator is selected from CDN1163, ranolazine, istaroxime, or gingerol. In another one aspect, the activator is adapted for oral, intravenous, intramuscular, cutaneous, subcutaneous, rectal, nasally, pulmonary, or transdermal administration.

In another embodiment, the present invention includes a method of treating cardiac function caused by a defect in the function of one or more sarco/endoplasmic reticulum Ca²⁺-ATPase (SERCA) pumps comprising: identifying a subject having a reduced cardiac function caused by a defect in the function of the one or more SERCA pumps; and providing the subject with an effective amount of an activator that enhances an activity of the one or more SERCA pumps. In one aspect, the reduced cardiac function is age-related. In another aspect, the reduced cardiac function is oxidative stress-related. In another aspect, the reduced cardiac function is in the left ventricle. In another aspect, the activator is selected from CDN1163, ranolazine, istaroxime, or gingerol. In another aspect, the one or more SERCA pumps is selected from SERCA1, 2, 3 or any isoforms of SERCA1, 2 or 3. In another one aspect, the activator is adapted for oral, intravenous, intramuscular, cutaneous, subcutaneous, rectal, nasally, pulmonary, or transdermal administration.

In yet another embodiment, the present invention includes a method of detecting and treating a subject with skeletal muscular atrophy caused by a defect in the function of sarco/endoplasmic reticulum Ca²⁺-ATPase (SERCA) pumps comprising: obtaining a muscle biopsy from the subject; detecting if the sample has decreased levels of SERCA pump activity; and treating the subject with an effective amount a SERCA activator that enhances and/or restores anactivity of the one or more SERCA pumps. In another one aspect, the activator is adapted for oral, intravenous, intramuscular, cutaneous, subcutaneous, rectal, nasally, pulmonary, or transdermal administration.

In another embodiment, the present invention includes a method of identifying a candidate agent for treating skeletal muscular atrophy caused by a defect in the function of one or more sarco/endoplasmic reticulum Ca²⁺-ATPase (SERCA) pumps, the method comprising: (a) contacting a mammalian cell with a test agent; (b) measuring an expression level and/or activity level of the one or more SERCA pumps in the mammalian cell relative to a reference value following the contacting; (c) determining that the test agent caused an increase in the expression level and/or activity level relative to the reference value; and (d) identifying the test agent as a candidate agent for treating skeletal muscular atrophy caused by a defect in the function of the one or more SERCA pumps. In another one aspect, the activator is adapted for oral, intravenous, intramuscular, cutaneous, subcutaneous, rectal, nasally, pulmonary, or transdermal administration.

In another embodiment, the present invention includes a method of treating a subject in need of at least one of: muscle regeneration, reduced muscle necrosis, improved mitochondrial morphology, extended lifespan, protection from contraction-induced injuries, or protection from Ca²⁺-driven necrosis in the gastrocnemius muscle, comprising providing the subject with an effective amount of an activator of one or more sarco/endoplasmic reticulum Ca2+-ATPase (SERCA) pumps sufficient to induce muscle regeneration, reduced muscle necrosis, improve mitochondrial morphology, extended lifespan, protect from contraction-induced injuries, or protect from Ca2+-driven necrosis in the gastrocnemius muscle. In one aspect, the candidate agent is a derivative of CDN1163, ranolazine, istaroxime, or gingerol. In another one aspect, the activator is adapted for oral, intravenous, intramuscular, cutaneous, subcutaneous, rectal, nasally, pulmonary, or transdermal administration.

### BRIEF DESCRIPTION OF THE DRAWINGS

For a more complete understanding of the features and advantages of the present invention, reference is now made to the detailed description of the invention along with the accompanying figures and in which:

FIGS. 1A to 1D show SERCA Ca²⁺ dependent ATPase activity and the expression of SERCA in the extracts from gastrocnemius muscles of ≈4 month old WT and Sod1^{-/-} mice treated with CDN1163 or vehicle for 7 weeks. CDN1163 restored the SERCA activity in the Sod1^{-/-} mice (FIG. 1A) as indicated by an increase in the maximum Ca²⁺ dependent SERCA activity (FIG. 1B). CDN1163 had no effect on the expression of SERCA isoforms at the mRNA (FIG. 1C) and protein (FIG. 1D) levels. Values are expressed as Mean±SEM. (n=5-6 per group). One-way ANOVA. * P≤0.05;

FIGS. 2A to 2C show CDN1163 restores muscle mass but not the body mass in the Sod1^{-/-} mice. Body mass (FIG. 2A), absolute gastrocnemius muscle mass (FIG. 2B) and the gastrocnemius mass normalized to body mass (FIG. 2C) from the WT and Sod1^{-/-} mice treated with CDN1163 or vehicle. Values are expressed as Mean±SEM. (n=6-10 per group). One-way ANOVA. * P≤0.05;

FIGS. 3A to 3C show contractile properties of the EDL muscles measured in the WT and Sod1^{-/-} mice treated with CDN1163 or vehicle. CDN1163 restored the specific force (FIG. 3A) in the Sod1^{-/-} mice without affecting the time to twitch peak tension (TTP) (FIG. 3B) and half relaxation time (½ R) (FIG. 3C). Values are expressed as Mean±SEM. (n=5-8 per group). One-way ANOVA. * P≤0.05, ** P≤0.01, *** P≤0.001;

FIGS. 4A and 4B show markers of oxidative stress in the WT and Sod1^{-/-} mice treated with CDN1163 or vehicle. CDN1163 reduces the state-1 and state-2 complex 1-linked (glutamate malate) mitochondrial H₂O₂ production (FIG. 4A) in the gastrocnemius muscles and the F2-isoprostanes as a marker of lipid peroxidation (FIG. 4B) in the quadriceps muscles in the in the Sod1^{-/-} mice when compared to the vehicle treatment. Values are expressed as Mean±SEM (n=8-12 per group). One-way ANOVA. * P≤0.05;

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

Sarcopenia, the progressive impairment in muscle mass and strength with aging, is a major contributor to frailty, loss of independent lifestyle and increased health care costs in the elderly (1). The loss of muscle quality is a universal and currently inevitable consequence of aging that has been shown to occur in humans and across vertebrate animal models (2). Despite the universality and negative consequences of sarcopenia, the precise underlying molecular mechanisms leading to muscle loss and dysfunction remain to be elucidated, and more importantly, no effective pharmacologic interventions have been established. Herein the inventors show their exciting findings supporting a pharmacologic intervention that is effective in reversing the sarcopenia phenotype in a mouse model of accelerated sarcopenia, the Sod1^{-/-} mice. Over the past several years, the inventor's laboratory has established the Sod1^{-/-} mouse as a model of age related sarcopenia. Sod1^{-/-} mice exhibit a number of phenotypes present in aging skeletal muscle including high levels of oxidative stress and damage, mitochondrial dysfunction and generation of ROS, loss of neuromuscular junction integrity and accelerated loss of muscle mass and weakness and have the added advantage that the majority of changes occur in mice less than 12 months of age (3-7). Thus, the Sod1^{-/-} mouse is an excellent model to test potential interventions for sarcopenia in relatively young mice.

The present inventors determined the effect of loss of sarco/endoplasmic reticulum Ca²⁺-ATPase (SERCA) activity is a critical determinant of sarcopenia using a mouse model of accelerated sarcopenia, mice lacking CuZnSOD (Sod1^{-/-} mice). SERCA activity is decreased by 27% in gastrocnemius muscle from Sod1^{-/-} mice compared to wild type mice. To determine whether activation of SERCA can reverse the sarcopenia phenotype in the Sod1^{-/-} mice, mice were treated for 7 weeks with CDN1163 (50 mg/kg, i.p., 3 times per week), a novel allosteric SERCA activator. Treatment with CDN1163 increased gastrocnemius muscle mass in Sod1^{-/-} mice by 23% and completely restored the 22% reduction in specific force measured in untreated Sod1^{-/-} versus wild type mice. CDN1163 also reversed the increase in mitochondrial ROS generation in the Sod1^{-/-} mice and reduced oxidative damage in muscle tissue measured as F2-isoprostanes by 50%. Collectively these findings show that reduced function of the SERCA pump contributes to muscle atrophy and reduced force generation, and that pharmacological stabilization of SERCA can reverse these effects, thus providing a powerful tool to counter age- and oxidative stress-associated muscle impairment.

A number of potential factors contributing to the underlying mechanisms of sarcopenia have been proposed, including oxidative stress, mitochondrial dysfunction, defects in muscle regeneration and impairments in calcium regulation and the muscle excitation-contraction (EC) coupling system (1, 8). EC coupling involves a series of molecular events that convert membrane depolarization into muscle contraction by releasing Ca²⁺ from the sarcoplasmic reticulum (SR) Ca²⁺ stores via the ryanodine receptors (RyRs). The subsequent reuptake of Ca2+ after contraction is executed by the SERCA pumps. Impaired function of the SERCA pump is associated with many chronic pathologies including aging (9), denervation (10) and muscular dystrophies (11). One potential molecular mechanism underlying this phenomenon may involve oxidative modification of SERCA and/or associated proteins. For example, treating isolated SR vesicles with peroxides causes oxidation and partial inactivation of SERCA pumps (12). SERCA protein oxidation and reduced activity have also been shown to occur in biological aging (13). Reduced SERCA function can result in cytoplasmic Ca²⁺ buildup leading to reduced muscle quality and/or quantity via activation of Ca²⁺ dependent proteases and mitochondrial dysfunction and ROS generation (14). Unlike prior art attempts to genetically manipulate SERCA; the inventors tested the ability of a pharmacological intervention to enhance muscle SERCA function using CDN1163, an allosteric activator of the SERCA pump. The inventors show for the first time that CDN1163 can increase muscle mass, restore muscle force and reduce mitochondrial ROS and oxidative stress in a mouse model of accelerated sarcopenia, the Sod1^{-/-} mice. The inventor's findings suggest that pharmacological activation of the SERCA pump may represent a promising therapy for sarcopenia (3, 5).

As used herein, the terms “sarco/endoplasmic (SR) reticulum Ca²⁺-ATPase”, or “SR Ca²⁺-ATPase” or “SERCA”, refers to a calcium ATPase-type P-ATPase. SERCA pumps reside in the sarcoplasmic reticulum (SR) within myocytes and are Ca²⁺ ATPases that transfers Ca²⁺ from the cytosol of the cell to the lumen of the SR by ATP hydrolysis during muscle relaxation. There are three major paralogs of SERCA, SERCA1, SERCA2, and SERCA3, which are variably expressed depending on the cell type.

As used herein, the term “SERCA activator” refers to a molecule that binds directly to SERCA or that allosterically increase its activity. Non-limiting examples of small molecule activators of the SERCA enzymes, such as SERCA1, 2, 3, or isotypes of SERCA1, 2, 3, include but are not limited to CDN1163, ranolazine, istaroxime, or gingerol, and precursors, active metabolites, or active derivatives thereof.

As used herein, the term “CDN1163” also known as 4-(1-Methylethoxy)-N-(2-methyl-8-quinolinyl)-benzamide, empirical formula C₂OH₂ON₂O₂, Molecular Weight 320.39, refers to an allosteric activator of SERCA.

As used herein, the terms “allosteric activator” refer to a compound that increases enzyme activity by binding of an effector at an allosteric site that affects binding or turnover a catalytic site. Within the scope of this invention such catalytic site occurs within the Ca²⁺-ATPase that transfers Ca²⁺ from the cytosol of the cell to the lumen of the SR at the expense of ATP hydrolysis during muscle relaxation.

A dosage unit for use of the one or more SERCA activator(s) of the present invention, may be a single compound or mixtures thereof with other compounds. The compounds may be mixed together in a manner that forms ionic or even covalent bonds. The SERCA activator(s) of the present invention may be administered in oral, intravenous (bolus or infusion), intraperitoneal, subcutaneous, or intramuscular form, all using dosage forms well known to those of ordinary skill in the pharmaceutical arts. Depending on the particular location or method of delivery, different dosage forms, e.g., tablets, capsules, pills, powders, granules, elixirs, tinctures, suspensions, syrups, and emulsions may be used to provide the one or more SERCA activator(s) are provided to a patient in need of therapy that includes the need for muscle regeneration, reduced muscle necrosis, improved mitochondrial morphology, extended lifespan and protection from contraction-induced injuries and Ca²⁺-driven necrosis in the gastrocnemius muscle. The SERCA activator(s) may also be administered as any one of known salt forms.

SERCA activator(s) is/are typically administered in admixture with suitable pharmaceutical salts, buffers, diluents, extenders, excipients and/or carriers (collectively referred to herein as a pharmaceutically acceptable carrier or carrier materials) selected based on the intended form of administration and as consistent with conventional pharmaceutical practices. Depending on the best location for administration, the SERCA activator(s) is/are may be formulated to provide, e.g., maximum and/or consistent dosing for the particular form for oral, rectal, topical, intravenous injection or parenteral administration. While the SERCA activator(s) is/are may be administered alone, it will generally be provided in a stable salt form mixed with a pharmaceutically acceptable carrier. The carrier may be solid or liquid, depending on the type and/or location of administration selected.

Techniques and compositions for selecting a dose and making useful dosage forms using the present invention are described in one or more of the following references: Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, N.Y., 1990; Katzung, ed., Basic and Clinical Pharmacology, Ninth Edition, McGraw Hill, 2007; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference, and the like, relevant portions incorporated herein by reference. The activator can be adapted for oral, intravenous, intramuscular, cutaneous, subcutaneous, rectal, nasally, pulmonary, or transdermal administration.

For example, the SERCA activator(s) may be included in a tablet. Tablets may contain, e.g., suitable binders, lubricants, disintegrating agents, coloring agents, flavoring agents, flow-inducing agents and/or melting agents. For example, oral administration may be in a dosage unit form of a tablet, gelcap, caplet or capsule, the active drug component being combined with an non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol, mixtures thereof, and the like. Suitable binders for use with the present invention include: starch, gelatin, natural sugars (e.g., glucose or beta-lactose), corn sweeteners, natural and synthetic gums (e.g., acacia, tragacanth or sodium alginate), carboxymethylcellulose, polyethylene glycol, waxes, and the like. Lubricants for use with the invention may include: sodium oleate, sodium stearate, magnesium stearate, sodium benzoate, sodium acetate, sodium chloride, mixtures thereof, and the like. Disintegrators may include: starch, methyl cellulose, agar, bentonite, xanthan gum, mixtures thereof, and the like.

SERCA activator(s) may be administered in the form of liposome delivery systems, e.g., small unilamellar vesicles, large unilamallar vesicles, and multilamellar vesicles, whether charged or uncharged. Liposomes may include one or more: phospholipids (e.g., cholesterol), stearylamine and/or phosphatidylcholines, mixtures thereof, and the like.

SERCA activator(s) may also be coupled to one or more soluble, biodegradable, bioacceptable polymers as drug carriers or as a prodrug. Such polymers may include: polyvinylpyrrolidone, pyran copolymer, polyhydroxylpropylmethacrylamide-phenol, polyhydroxyethylasparta-midephenol, or polyethyleneoxide-polylysine substituted with palmitoyl residues, mixtures thereof, and the like. Furthermore, the SERCA activator(s) may be coupled with one or more biodegradable polymers to achieve controlled release of the SERCA activator(s), biodegradable polymers for use with the present invention include: polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels, mixtures thereof, and the like.

In one embodiment, gelatin capsules (gelcaps) may include the SERCA activator(s) and powdered carriers, such as lactose, starch, cellulose derivatives, magnesium stearate, stearic and the like. Like diluents may be used to make compressed tablets. Both tablets and capsules may be manufactured as immediate-release, mixed-release or sustained-release formulations to provide for a range of release of medication over a period of minutes to hours. Compressed tablets may be sugar coated or film coated to mask any unpleasant taste and protect the tablet from the atmosphere. An enteric coating may be used to provide selective disintegration in, e.g., the gastrointestinal tract.

For oral administration in a liquid dosage form, the oral drug components may be combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents, mixtures thereof, and the like.

Liquid dosage forms for oral administration may also include coloring and flavoring agents that increase patient acceptance and therefore compliance with a dosing regimen. In general, water, a suitable oil, saline, aqueous dextrose (e.g., glucose, lactose and related sugar solutions) and glycols (e.g., propylene glycol or polyethylene glycols) may be used as suitable carriers for parenteral solutions. Solutions for parenteral administration include generally, a water-soluble salt of the active ingredient, suitable stabilizing agents, and if necessary, buffering salts. Antioxidizing agents such as sodium bisulfite, sodium sulfite and/or ascorbic acid, either alone or in combination, are suitable stabilizing agents. Citric acid and its salts and sodium EDTA may also be included to increase stability. In addition, parenteral solutions may include pharmaceutically acceptable preservatives, e.g., benzalkonium chloride, methyl- or propyl-paraben, and/or chlorobutanol. Suitable pharmaceutical carriers are described in Remington's Pharmaceutical Sciences, Mack Publishing Company, a standard reference text in this field, relevant portions incorporated herein by reference.

For direct delivery to the nasal passages, sinuses, mouth, throat, esophagous, tachea, lungs and alveoli, the SERCA activator(s) may also be delivered as an intranasal form via use of a suitable intranasal vehicle. For dermal and transdermal delivery, the SERCA activator(s) may be delivered using lotions, creams, oils, elixirs, serums, transdermal skin patches and the like, as are well known to those of ordinary skill in that art. Parenteral and intravenous forms may also include pharmaceutically acceptable salts and/or minerals and other materials to make them compatible with the type of injection or delivery system chosen, e.g., a buffered, isotonic solution. Examples of useful pharmaceutical dosage forms for administration of SERCA activator(s) may include the following forms.

Capsules. Capsules may be prepared by filling standard two-piece hard gelatin capsules each with 10 to 500 milligrams of powdered active ingredient, 5 to 150 milligrams of lactose, 5 to 50 milligrams of cellulose and 6 milligrams magnesium stearate.

Soft Gelatin Capsules. A mixture of active ingredient is dissolved in a digestible oil such as soybean oil, cottonseed oil or olive oil. The active ingredient is prepared and injected by using a positive displacement pump into gelatin to form soft gelatin capsules containing, e.g., 100-500 milligrams of the active ingredient. The capsules are washed and dried.

Tablets. A large number of tablets are prepared by conventional procedures so that the dosage unit was 100-500 milligrams of active ingredient, 0.2 milligrams of colloidal silicon dioxide, 5 milligrams of magnesium stearate, 50-275 milligrams of microcrystalline cellulose, 11 milligrams of starch and 98.8 milligrams of lactose. Appropriate coatings may be applied to increase palatability or delay absorption.

To provide an effervescent tablet, appropriate amounts of, e.g., monosodium citrate and sodium bicarbonate are blended together and then roller compacted, in the absence of water, to form flakes that are then crushed to give granulates. The granulates are then combined with the active ingredient, drug and/or salt thereof, conventional beading or filling agents and, optionally, sweeteners, flavors and lubricants.

Injectable solution. A parenteral composition suitable for administration by injection is prepared by stirring 1.5% by weight of active ingredient in deionized water and mixed with, e.g., up to 10% by volume propylene glycol and water. The solution is made isotonic with sodium chloride and sterilized using, e.g., ultrafiltration.

Suspension. An aqueous suspension is prepared for oral administration so that each 5 ml contain 100 mg of finely divided active ingredient, 200 mg of sodium carboxymethyl cellulose, 5 mg of sodium benzoate, 1.0 g of sorbitol solution, U.S.P., and 0.025 ml of vanillin.

For mini-tablets, the active ingredient is compressed into a hardness in the range 6 to 12 Kp. The hardness of the final tablets is influenced by the linear roller compaction strength used in preparing the granulates, which are influenced by the particle size of, e.g., the monosodium hydrogen carbonate and sodium hydrogen carbonate. For smaller particle sizes, a linear roller compaction strength of about 15 to 20 KN/cm may be used.

Kits. The present invention also includes pharmaceutical kits useful, for example, for the treatment of cancer, which comprise one or more containers containing a pharmaceutical composition comprising a therapeutically effective amount of the SERCA activator(s). Such kits may further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, may also be included in the kit. It should be understood that although the specified materials and conditions are important in practicing the invention, unspecified materials and conditions are not excluded so long as they do not prevent the benefits of the invention from being realized.

Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

As used herein, the term “chewable” refers to the SERCA activator(s) formulated into semi-soft, palatable and stable chewable treats for use without the addition of water. It should be appreciated to the skilled artisan that a chewable composition will be stable and palatable, fast disintegrating, semi-soft medicated chewable tablets (treats) by extrusion without the addition of extraneous water. A soft chewable tablets does not harden on storage and are resistant to microbial contamination. A semi-soft chewable contain a blend of any one or more of binders, flavours, palatability enhancers, humectants, disintegrating agents, non-aqueous solvents, and diluents that are plasticized with liquid plasticizers, such as glycols and polyols to make them ductile and extrudable. The chewbale can be made by extrusion, e.g., including fats or lipids as plasticizers and binding agents, is manufactured in the absence of added water, uses plasticizers to replace water in extrudable matrices, contains humectants to maintain the extrudable chew in a pliant and soft state during its shelf life, or any combination thereof. The chewable form may be provided in conjunction with one or more flavorants and/or taste masking agents that improve the taste of the formulation greater than 10, 20, 30, 40, 50, 60, 70, 80, or 90%. The chewable can include the active agent and the ion exchange resin to enhance taste masking.

In certain embodiments, the SERCA activator(s) of the present invention can be formulated into a dosage form in which the final formulation includes no other active agent. In such an embodiment, the SERCA activator(s) is/are provided such that only non-active excipients, carriers, etc., that are pharmacologically acceptable and without any other active agent, which shall be used with the phrase “consisting essentially of” requires the specified integer(s) or steps as well as those that do not materially affect the character or function of the claimed invention. In other embodiments, the SERCA activator(s) may be the only active agent provided in a simple carrier, in which case the composition is said to “consist” of the SERCA activator(s) without any other agents, and when used in a method, the SERCA activator(s) will be said to be included in a formulation “consisting” of the active SERCA activator(s).

Animals. The generation and characterization of the Sod1^{-/-} mice is described in detail elsewhere (3, 24). Female ≈2 months old C57BL/6J wild-type (WT; n=12-18) and Sod1^{-/-} mice (n=12-18) were divided into four groups and treated with either vehicle (10% DMSO, 10% Tween-80 in PBS) or CDN1163 (50 mg/kg) by intraperitoneal injection three times per week for 7 weeks. Care and management of the mice was executed according to The Guide for the Care and Use of Laboratory Animals and approved by the institutional Animal Care and Use Committee at the Oklahoma Medical Research Foundation (OKC, Okla., USA).

Protein preparation and western blot. Muscles were homogenized in RIPA buffer containing 50 mM Tris (pH 7.4), 150 mM NaCl, and protease inhibitors. Protein was quantified using the Bio-Rad kit (Sigma-Aldrich, Poole, UK) and transferred to nitrocellulose membrane after electrophoresis using 8-15% polyacrylamide gels. Bands were scanned and quantified using Gene Tool system (SynGene—Frederick, Md.). All image intensities were normalized to protein intensity based on the ponceau stain.

Analysis of oxidative damage by F2-isoprostane level. The levels of F2-isoprostanes in quadriceps muscle were measured using thin layer chromatography and GC-mass spectrometry as described previously (25).

Quantification of mRNA levels using real-time PCR. Total RNA was extracted using TRI reagent and the cDNA was prepared from 1 mg of the total RNA using iScript™ cDNA

Synthesis kit (Bio-Rad, Hercules, Calif., USA). 2.5 ng of cDNA samples were amplified using specific primers along with fast SYBR green master mix (Applied Biosystems, Grand Island, N.Y., USA). The data were analyzed using the ΔΔCt method.

Contractile Measurements. Contractile force generation was measured in isolated extensor digitorum longus (EDL) muscle using a 1200A in vitro test system (Aurora Scientific Inc.) as described elsewhere (7). Briefly, muscles were individually tied to a model 300C servomotor (Aurora Scientific Inc.) and fixed within a water bath containing an oxygenated (95% O₂, 5% CO₂) Krebs-Ringer solution (in mM: 137 NaCl, 5 KCl, 1 MgSO₄, 1 NaH₂PO₄, 24 NaHCO₃, 2 CaCl₂) maintained at 32 C. Computer controlled stimulation was applied through a model 701C stimulator (Aurora Scientific Inc.). Force frequency curves were generated with stimulation frequencies between 1 and 300 Hz, while the fatigue protocol consisted of repeated 150 Hz stimuli, every 5 s for 400 s. All data were recorded and analyzed using commercial software (DMC and DMA, Aurora Scientific). Specific force (N/cm²) was measured using muscle length and mass.

SERCA activity. The measurement of SERCA activity was performed in the muscle homogenates at 37° C. using a spectrophotometric assay as previously described (26).

Mitochondrial function. Mitochondrial isolation and assays for H₂O₂ generation were performed in gastrocnemius muscle as described previously by the inventor's laboratory (27). Freshly isolated mitochondria were used for H₂O₂ release assay using the Amplex red-HRP method. Isolated mitochondrial are exposed to respiratory substrates and HRP (1 U Ml-1) in the assay buffer catalyzes the H₂O₂-dependent oxidation of non-fluorescent Amplex Red (80 μM) (Molecular Probes, Eugene, Oreg., USA) to fluorescent resofurin red (excitation λ=544 nm, emission λ=590 nm at 37° C.). The slope of the increase in fluorescence was converted to the rate of H₂O₂ production with standard curve.

Statistical Analysis. Data are presented as mean±SEM and the comparisons among the four groups were performed by one-way analysis of variance (ANOVA). Data were analyzed using GraphPad Prism 7 and the p values of less than 0.05 were considered statistically significant.

This is the first study showing that the direct pharmacological activation of skeletal muscle SERCA by the SERCA activator CDN1163 rescues muscle atrophy and weakness in a mouse model of sarcopenia. Herein the inventors report that SERCA activation is associated with the restoration of muscle mass and force and prevention of mitochondrial ROS generation and oxidative damage in the Sod1^{-/-} mice. These findings show that the pharmacological restoration of SERCA may be a powerful intervention to prevent oxidative stress-mediated muscle impairment during aging and diseases involving muscular degeneration.

SERCA plays a critical role in cellular maintenance of calcium levels. This is especially important in skeletal muscle where calcium levels are elevated following muscle contraction and need to be repeatedly restored to resting levels to avoid deleterious effects of high calcium. There are two major SERCA isoforms in mouse hind limb muscles, SERCA1a the more abundant fast twitch isoform and SERCA2a the relatively low abundance slow twitch isoform. Together they account for ≥70% of Ca²⁺ removal from the cytosol (18). Reduced SERCA ATPase activity is associated with aging muscle (9), denervation (10) and muscular dystrophies (19). Thus, the present invention shows for the first time that the restoration of SERCA function using a therapeutic agent can be used to treat and to prevent muscle defects.

The inventor's laboratory has extensively studied mechanisms of sarcopenia using the Sod1^{-/-} mouse model that shows an accelerated appearance of a number of phenotypes associated with aging skeletal muscle, including loss of muscle mass and force. The inventors found that SERCA activity is decreased in gastrocnemius muscle in the Sod1^{-/-} mice, consistent with previous reports of reduced SERCA activity in aging muscle and denervation (9, 10). SERCA has been shown to be inactivated by elevated oxidative stress, thus it is possible that the reduction in SERCA activity in the Sod1^{-/-} mice is related to oxidative inactivation of the enzyme. In support of this, the amino acids peroxides are shown to selectively oxidize cysteine residues of SERCA and partially inactivate the pump in the isolated SR preparations (12). The inventors recognized that high levels of oxidative stress and mitochondrial dysfunction have previously been linked to elevated cytosolic calcium levels (14). The inventors show herein that activation and maintenance of SERCA activity by CDN1163 maintains physiologic calcium levels and prevent or reduce muscle atrophy and weakness in the Sod1^{-/-} mice. Interestingly, CDN1163 was recently shown to prevent ER stress and have beneficial effects in hepatic (17), pancreatic (21) and neuronal (22) tissues. The inventors show herein that CDN1163 restored SERCA activity to levels found in wild type mice, reversed mitochondrial ROS generation, prevented oxidative damage and protected from muscle atrophy and loss of contractile force generation.

By way of explanation, and in no way a limitation of the present invention, the protective effects of CDN1163 on muscle mass and function may be due to the maintenance of calcium levels and/or an indirect effect of the compound on mitochondrial function and downstream oxidative stress that can propagate muscle degenerative processes. Mitochondria are an important source of ROS in the skeletal muscle, and the local structural and functional communications between SR and mitochondria are well characterized (16). It is possible that SERCA dysfunction and mitochondrial ROS production are locally amplified leading to progressive muscle impairment. Restoration of SERCA activity has been shown to reduce mitochondrial swelling and free radical production in the gastrocnemius muscles of mice with muscular dystrophy (19). It is possible that the restoration of SERCA activity could prevent mitochondrial-derived oxidative stress and therefore mitigate the disease phenotype in the Sod1^{-/-} mice. The reduced emission of mitochondrial H₂O₂ the inventors measured in the CDN1163 treated Sod1^{-/-} mice is consistent with this idea.

Apart from inducing mitochondrial damage, SERCA pump dysfunction can also disrupt Excitation-Contraction (EC) coupling machinery leading to reduced muscle strength as the mice with decrease in SERCA pump activity show reduced force-generating capacity (19). The inventors can propose at least two ways that the disrupted EC coupling and the associated oxidative stress might contribute to muscle weakness in the Sod1^{-/-} mice. It is possible that the Ca²⁺ dysregulation and increased ROS production reduce Ca²⁺ sensitivity of the contractile apparatus. In support of this, a direct coupling between increased ROS and myofibrillar Ca²⁺ sensitivity is proposed leading to reduced force production in the conditions of increased oxidative stress (23). Alternatively, increased ROS may damage SR Ca²⁺ release channels RyRs, resulting in reduced Ca release during contraction and contributing to muscle weakness. Hence the SERCA restoration-mediated improved Ca²⁺ handling and reduced ROS production in the CDN1163 treated Sod1^{-/-} mice might be contributing to restored force-generating capacity.

In conclusion, the inventors validate for the first time that the pharmacological activation of SERCA is a powerful intervention to prevent muscle impairment associated with the sarcopenia phenotype. Importantly, CDN1163 restores oxidative balance, force-generating capacity and the muscle mass by reversing the loss of SERCA function in the Sod1^{-/-} mice. Currently, there are no known drugs to effectively offset muscle weakness and atrophy. This study provides an important proof-of-concept that the CDN1163 has the potential to become an effective therapy for age- and oxidative stress-related muscle diseases.

### EXAMPLES

The following examples are included to further illustrate various aspects of the invention. It should be appreciated by those of skill in the art that the techniques disclosed in the examples, which follow, represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

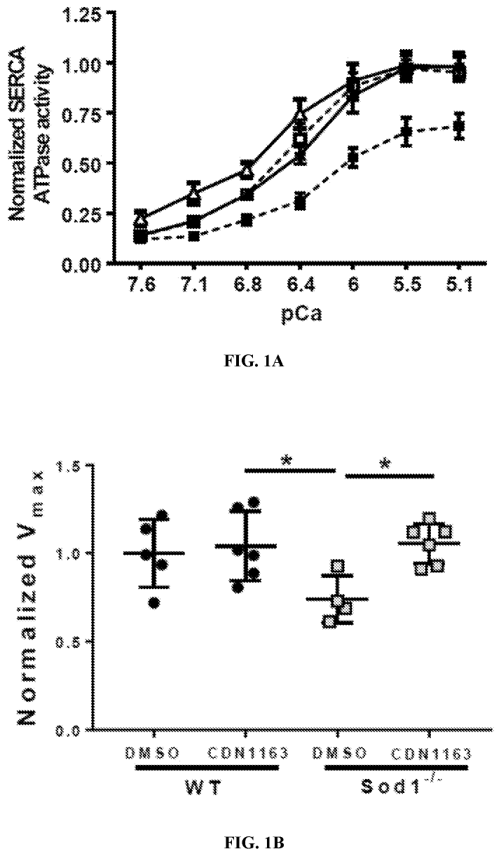

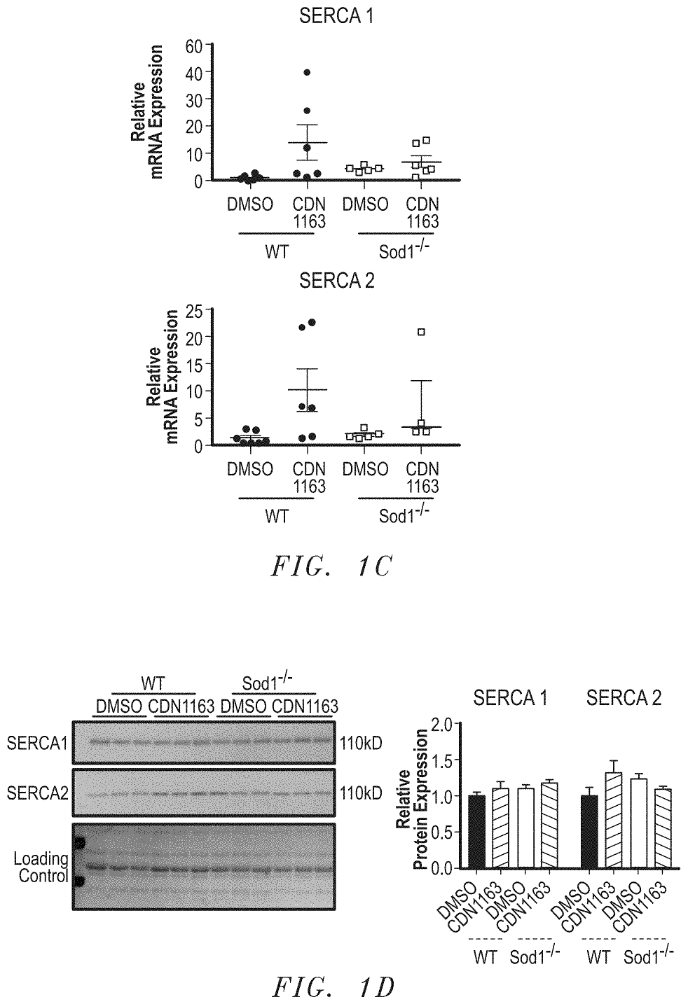

CDN1163 rescues the SERCA activity in the Sod1^{-/-} mice. Ca²⁺-dependent Ca²⁺-ATPase activity was measured in gastrocnemius muscle homogenates to determine the effects of CDN1163 on SERCA function. Maximum Ca²⁺ dependent (SR) Ca²⁺-ATPase is significantly reduced (≈33%; p<0.001) in muscle homogenates from the Sod1^{-/-} mice when compared to WT mice. The reduction in SERCA activity is completely restored with 7 weeks of CDN1163 treatment (FIGS. 1A, 1B). Levels of SERCA1 and SERCA2 mRNA and protein were not changed in Sod1^{-/-} mice and were not altered by CDN1163 treatment (FIGS. 1C, 1D).

### Example 2

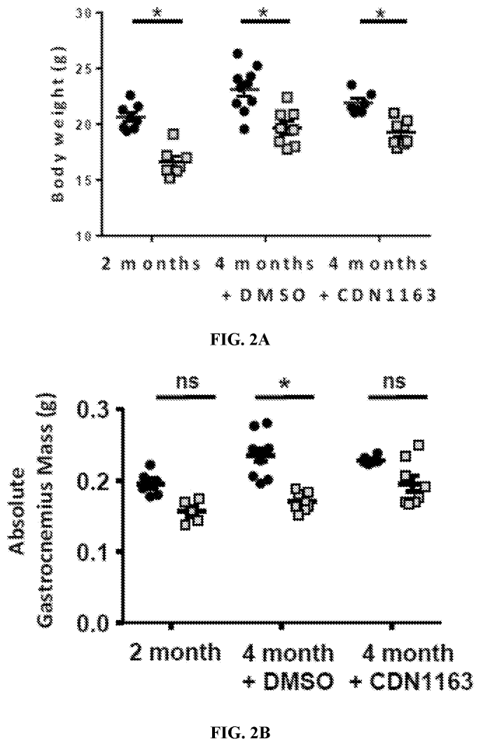

CDN1163 prevents gastrocnemius muscle atrophy in the Sod1^{-/-} mice. At 2 months of age (the starting point for this study) the Sod1^{-/-} mice have a significantly smaller body mass (˜19% less than age matched WT mice; 20.6±0.4 versus 16.6±0.4) as the inventors have previously reported (3, 7) (FIG. 2A). CDN1163 treatment for 7 weeks did not alter body weight in WT or Sod1^{-/-} mice, and the Sod1^{-/-} mice remained approximately 14% smaller than the WT mice at the end of the 7 week treatment period. At 2 months of age, neither absolute (FIG. 2B) nor normalized (FIG. 2C) gastrocnemius muscle mass is statistically lower in Sod1^{-/-} versus WT female mice. However, at the end of the 7 week period (˜4 months of age) there is a significant decrease in both absolute and normalized gastrocnemius muscle mass in the untreated Sod1^{-/-} mice compared to untreated WT mice (FIGS. 2B, 2C) consistent with the inventor's previous reports(3). Remarkably, 7 weeks of CDN1163 treatment completely prevented this atrophy in the Sod1^{-/-} mice, and the normalized gastrocnemius mass of CDN treated Sod1^{-/-} mice is 23% greater than Sod1^{-/-} untreated mice (FIG. 2C).

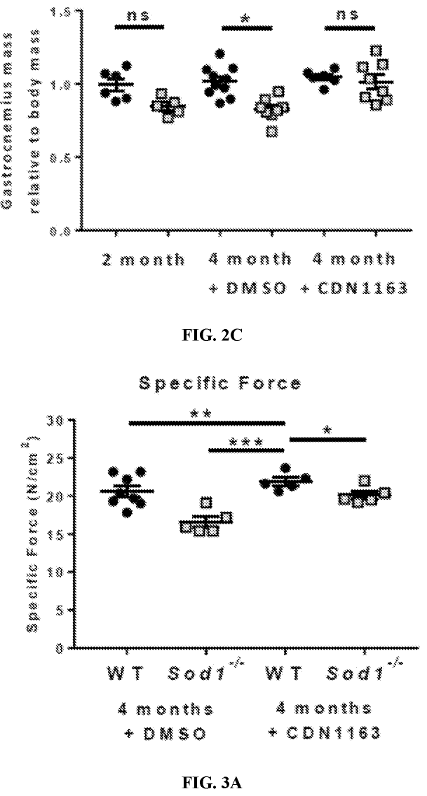

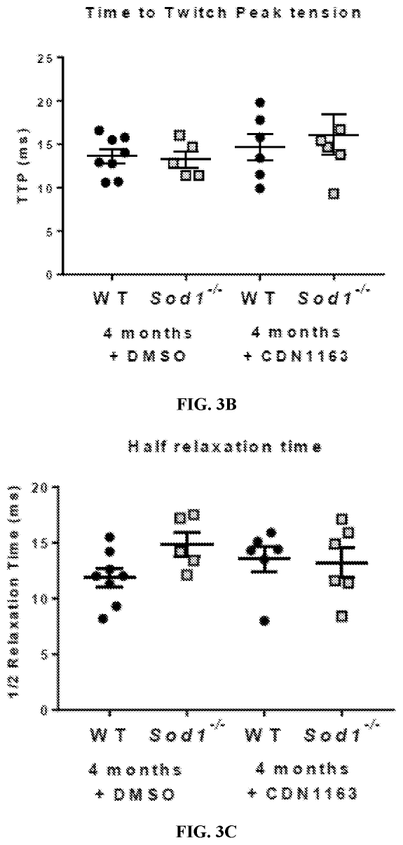

Example 3. CDN1163 prevents contractile dysfunction in the Sod1^{-/-} mice. In agreement with the inventor's previous findings, the inventors measured a significant decline in the in-vitro specific force measured in EDL muscle of Sod1^{-/-} mice (˜19%, p<0.05) when compared to WT mice (FIG. 3A). Consistent with the maintenance of muscle mass, 7 weeks of CDN1163 treatment completely restored the specific force in the Sod1^{-/-} mice. Importantly, these changes occur in vitro independent of muscle mass and innervation status suggesting a beneficial effect of CDN1163 on intrinsic force-generating properties of the EDL muscle. On the other hand, CDN1163 has no effect on the time to peak contraction (TTP) and half relaxation time (RT1/2) in the EDL muscle during twitch contractions (FIGS. 3B, 3C).

### Example 5

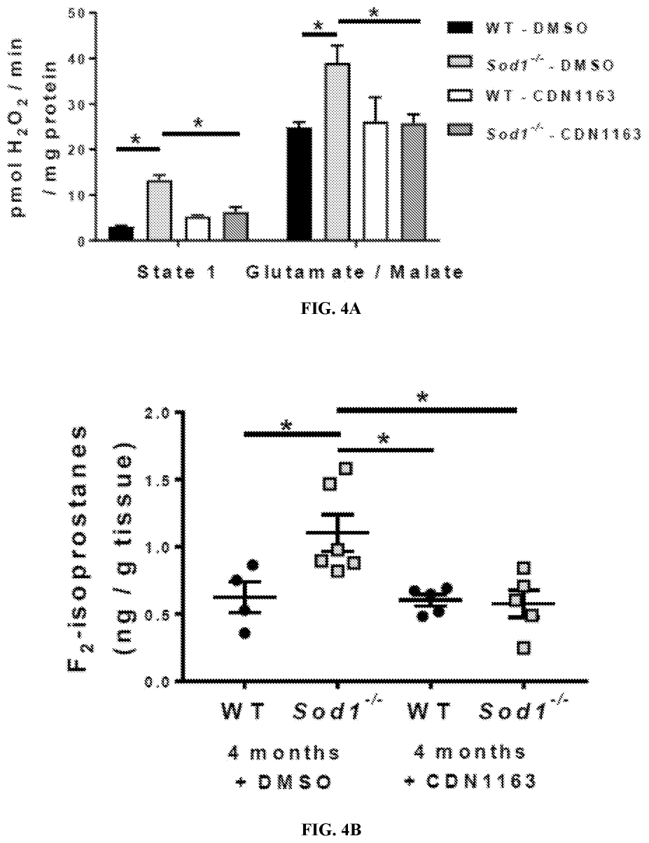

CDN1163 attenuates mitochondrial dysfunction in the Sod1^{-/-} mice. It is well documented that elevated levels of cytosolic Ca²⁺ lead to increased mitochondrial ROS production (16). The inventors have previously shown that the mitochondria from the Sod1^{-/-} mice show structural and functional defects (4, 5). To test whether the activation of SERCA pump function improves mitochondrial function, the inventors measured mitochondrial ROS production as H₂O₂ emission using isolated mitochondria from the gastrocnemius muscle. In accordance with the inventor's previous findings, mitochondria from Sod1^{-/-} mice showed significantly greater (≈340%, p<0.001) H₂O₂ production in State-1 respiration (mitochondria respiring without addition of external substrate), than mitochondria from gastrocnemius muscle from WT mice (FIG. 4A). In contrast, isolated mitochondria from CDN treated muscle from Sod1^{-/-} mice do not show elevated levels of H₂O₂ production, i.e., levels are similar to mitochondria from WT mice. When respiratory substrates glutamate/malate are added to stimulate electron flow through Complex I, mitochondrial H₂O₂ production is still significantly higher in the Sod1^{-/-} mice (≈56%, p<0.05) compared to WT mice. CDN1163 treatment in the Sod1^{-/-} mice returns glutamate/malate stimulated H₂O₂ generation to WT levels (FIG. 4A).

### Example 6

CDN1163 reduces the oxidative damage in the Sod1^{-/-} mice. The inventors have previously reported increased oxidative damage in the skeletal muscles of Sod1^{-/-} mice (6) that might be contributing to muscle atrophy and weakness. CDN1163 was shown to reduce plasma levels of malondialdehyde, a marker of oxidative stress, in the ob/ob mouse model (17). Therefore the inventors investigated whether CDN1163 can prevent oxidative stress-induced damage by measuring the levels of F2-isoprostanes, an indicator of lipid peroxidation, in the quadriceps muscles. As shown in FIG. 4B, the F2-isoprostanes levels in quadriceps muscle are significantly higher (≈76%, p<0.001) in the Sod1^{-/-} mice compared to WT mice. However, CDN1163 treatment prevented this increase in the Sod1^{-/-} mice.

### Example 7

A method of identifying a candidate agent for treating skeletal muscular atrophy caused by a defect in the function of one or more sarco/endoplasmic reticulum Ca²⁺-ATPase (SERCA) pumps, the method comprising: (a) contacting a mammalian cell with a test agent; (b) measuring an expression level and/or activity level of the one or more SERCA pumps in the mammalian cell relative to a reference value following the contacting; (c) determining that the test agent caused an increase in the expression level and/or activity level relative to the reference value; and (d) identifying the test agent as a candidate agent for treating skeletal muscular atrophy caused by a defect in the function of the one or more SERCA pumps.

### Example 8

A method of treating a subject in need of at least one of: muscle regeneration, reduced muscle necrosis, improved mitochondrial morphology, extended lifespan, protection from contraction-induced injuries, or protection from Ca²⁺-driven necrosis in the gastrocnemius muscle, comprising providing the subject with an effective amount of an activator of one or more sarco/endoplasmic reticulum Ca²⁺-ATPase (SERCA) pumps sufficient to induce muscle regeneration, reduced muscle necrosis, improve mitochondrial morphology, extended lifespan, protect from contraction-induced injuries, or protect from Ca2+-driven necrosis in the gastrocnemius muscle. In one aspect, the candidate agent is a derivative of CDN1163, ranolazine, istaroxime, or gingerol.

### Example 9. Aging Study

The present inventors studied a group of old wildtype mice (24-26 months) with and without 8-10 months treatment with CDN1163. The old wild-type mice had a significantly smaller body weight (≈11%, p<0.05) when compared to the 16 month old baseline control group for this study. Treatment with CDN1163 did not prevent the reduction in body weight in the old mice. Further, the old mice show significant atrophy of the gastrocnemius muscles when compared to baseline group (≈10%, p<0.05). However, treatment with CDN1163 completely prevented the age-related atrophy of the gastrocnemius muscles in these mice. The inventors also measured the muscle force generating capacity in the mouse EDL muscles because of large body of literature showing reduced strength of extensor digitorum longus (EDL) muscle with aging. EDL muscle weakness in the old wild-type mice (≈11%, p<0.05) was consistent with previous studies, when compared to the baseline group. Treatment with CDN1163 prevented this decline in force in the old mice, which showed no significant muscle weakness when compared to baseline group. This effect was consistent at stimulation frequencies for maximal and sub-maximal (≈50% of maximal force) tetanic forces normalized for muscle mass. On the other hand, CDN1163 had no significant effects on muscle half relaxation time and time to maximal twitch force, which are the measures of muscle calcium transience and fiber-type compositions. As such, CDN1163 was able to reduce muscle weakness, while not having side-effects related to muscle half relaxation time and time to maximal twitch force.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112 as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. Ali S & Garcia J M (2014) Sarcopenia, cachexia and aging: diagnosis, mechanisms and therapeutic options—a mini-review. Gerontology 60(4):294-305.
2. Ishida J, et al. (2017) Animal models of cachexia and sarcopenia in chronic illness: Cardiac function, body composition changes and therapeutic results. Int J Cardiol 238:12-18.
3. Muller F L, et al. (2006) Absence of CuZn superoxide dismutase leads to elevated oxidative stress and acceleration of age-dependent skeletal muscle atrophy. Free Radic Biol Med 40(11): 1993-2004.
4. Jang Y C, et al. (2012) Dietary restriction attenuates age-associated muscle atrophy by lowering oxidative stress in mice even in complete absence of CuZnSOD. Aging Cell 11(5):770-782.
5. Jang Y C, et al. (2010) Increased superoxide in vivo accelerates age-associated muscle atrophy through mitochondrial dysfunction and neuromuscular junction degeneration. FASEB J 24(5):1376-1390.
6. Sakellariou G K, et al. (2011) Role of superoxide-nitric oxide interactions in the accelerated age-related loss of muscle mass in mice lacking Cu,Zn superoxide dismutase. Aging Cell 10(5):749-760.
7. Larkin L M, et al. (2011) Skeletal muscle weakness due to deficiency of CuZn-superoxide dismutase is associated with loss of functional innervation. Am J Physiol Regul Integr Comp Physiol 301(5):R1400-1407.
8. Boncompagni S, d'Amelio L, Fulle S, Fano G, & Protasi F (2006) Progressive disorganization of the excitation-contraction coupling apparatus in aging human skeletal muscle as revealed by electron microscopy: a possible role in the decline of muscle performance. J Gerontol A Biol Sci Med Sci 61(10):995-1008.
9. Thomas M M, Khan W, Betik A C, Wright K J, & Hepple R T (2010) Initiating exercise training in late middle age minimally protects muscle contractile function and increases myocyte oxidative damage in senescent rats. Exp Gerontol 45(11):856-867.
10. Dufresne S S, et al. (2016) Muscle RANK is a key regulator of Ca2+ storage, SERCA activity, and function of fast-twitch skeletal muscles. Am J Physiol Cell Physiol 310(8):C663-672.
11. Voit A, et al. (2017) Reducing sarcolipin expression mitigates Duchenne muscular dystrophy and associated cardiomyopathy in mice. Nat Commun 8(1):1068.
12. Dremina E S, Sharov V S, Davies M J, & Schoneich C (2007) Oxidation and inactivation of SERCA by selective reaction of cysteine residues with amino acid peroxides. Chem Res

Toxicol 20(10): 1462-1469.
13. Sharov V S, Dremina E S, Galeva N A, Williams T D, & Schoneich C (2006) Quantitative mapping of oxidation-sensitive cysteine residues in SERCA in vivo and in vitro by HPLC-electrospray-tandem MS: selective protein oxidation during biological aging. Biochem J 394(Pt 3):605-615.
14. Vallejo-Illarramendi A, Toral-Ojeda I, Aldanondo G, & Lopez de Munain A (2014) Dysregulation of calcium homeostasis in muscular dystrophies. Expert Rev Mol Med 16:e16.
15. Mazala D A, et al. (2015) SERCA1 overexpression minimizes skeletal muscle damage in dystrophic mouse models. Am J Physiol Cell Physiol 308(9):C699-709.
16. Eisner V, Csordas G, & Hajnoczky G (2013) Interactions between sarco-endoplasmic reticulum and mitochondria in cardiac and skeletal muscle—pivotal roles in Ca(2)(+) and reactive oxygen species signaling. J Cell Sci 126(Pt 14):2965-2978.
17. Kang S, et al. (2016) Small Molecular Allosteric Activator of the Sarco/Endoplasmic Reticulum Ca2+-ATPase (SERCA) Attenuates Diabetes and Metabolic Disorders. J Biol Chem 291(10):5185-5198.
18. Periasamy M & Kalyanasundaram A (2007) SERCA pump isoforms: their role in calcium transport and disease. Muscle Nerve 35(4):430-442.
19. Goonasekera S A, et al. (2011) Mitigation of muscular dystrophy in mice by SERCA overexpression in skeletal muscle. J Clin Invest 121(3):1044-1052.
20. Gehrig S M, et al. (2012) Hsp72 preserves muscle function and slows progression of severe muscular dystrophy. Nature 484(7394):394-398.
21. Tong X, et al. (2016) SERCA2 Deficiency Impairs Pancreatic beta-Cell Function in Response to Diet-Induced Obesity. Diabetes 65(10):3039-3052.
22. Dahl R (2017) A new target for Parkinson's disease: Small molecule SERCA activator CDN1163 ameliorates dyskinesia in 6-OHDA-lesioned rats. Bioorg Med Chem 25(1):53-57.
23. Moopanar T R & Allen D G (2005) Reactive oxygen species reduce myofibrillar Ca2+ sensitivity in fatiguing mouse skeletal muscle at 37 degrees C. J Physiol 564(Pt 1):189-199.
24. Elchuri S, et al. (2005) CuZnSOD deficiency leads to persistent and widespread oxidative damage and hepatocarcinogenesis later in life. Oncogene 24(3):367-380.
25. Zhang Y, et al. (2013) Dietary restriction attenuates the accelerated aging phenotype of Sod1 (−/−) mice. Free radical biology & medicine 60:300-306.
26. Fu M H & Tupling A R (2009) Protective effects of Hsp70 on the structure and function of SERCA2a expressed in HEK-293 cells during heat stress. American journal of physiology. Heart and circulatory physiology 296(4):H1175-1183.
27. Chen L, et al. (2008) Reduction of mitochondrial H₂O₂ by overexpressing peroxiredoxin 3 improves glucose tolerance in mice. Aging Cell 7(6):866-878.

1. A method of treating a skeletal muscular atrophy or skeletal muscular degeneration caused by a defect in the function of one or more sarco/endoplasmic reticulum Ca²⁺-ATPase (SERCA) pumps comprising:
identifying a subject having a muscular atrophy or muscular degeneration caused by a defect in the function of the one or more SERCA pumps; and
providing the subject with an effective amount of an activator that enhances an activity of the one or more SERCA pumps.

2. The method of claim 1, wherein the skeletal muscular atrophy is age-related, oxidative stress-related, or is sacropenia.

3. (canceled)

4. (canceled)

5. The method of claim 1, wherein the SERCA pump is selected from SERCA1, 2, 3 or any isoforms of SERCA1, 2 or 3.

6. The method of claim 1, wherein the activator is selected from CDN1163, ranolazine, istaroxime, or gingerol.

7. The method of claim 1, wherein the activator is adapted for oral, intravenous, intramuscular, cutaneous, subcutaneous, rectal, nasally, pulmonary, or transdermal administration.

8. (canceled)

9. The method of claim 1, wherein the skeletal muscular degeneration is age-related, oxidative stress-related, or is muscular atrophy.

10. (canceled)

11. (canceled)

12. The method of claim 9, wherein the muscular atrophy is age-related or oxidative stress-related.

13. (canceled)

14. (canceled)

15. (canceled)

16. A method of treating muscular dystrophy caused by a defect in the function of one or more sarco/endoplasmic reticulum Ca²⁺-ATPase (SERCA) pumps comprising:
identifying a subject having a denervation caused by a defect in the function of the one or more SERCA pumps; and
providing the subject with an effective amount of an activator that enhances an activity of the one or more SERCA pumps.

17. The method of claim 16, wherein the muscular dystrophy is age-related, oxidative stress-related, is Duchenne muscular dystrophy or the muscular dystrophy is muscular atrophy that is age-related, or oxidative stress-related.

18. (canceled)

19. (canceled)

20. (canceled)

21. (canceled)

22. (canceled)

23. The method of claim 16 wherein the SERCA pumps is selected from SERCA1, 2, 3 or any isoforms of SERCA1, 2 or 3.

24. The method of claim 16, wherein the activator is selected from CDN1163, ranolazine, istaroxime, or gingerol.

25. A method of treating denervation caused by a defect in the function of one or more sarco/endoplasmic reticulum Ca²⁺-ATPase (SERCA) pumps comprising:
identifying a subject having a denervation caused by a defect in the function of the one or more SERCA pumps; and
providing the subject with an effective amount of an activator that enhances an activity of the one or more SERCA pumps.

26. The method of claim 25, wherein the denervation is symptomatic of hyperthyroidism.

27. The method of claim 25, wherein the denervation is age-related, or oxidative stress-related.

28. (canceled)

29. The method of claim 25, wherein the one or more SERCA pumps is selected from SERCA1, 2, 3 or any isoforms of SERCA1, 2 or 3.

30. The method of claim 25, wherein the activator is selected from CDN1163, ranolazine, istaroxime, or gingerol.

31. A method of treating cardiac function caused by a defect in the function of one or more sarco/endoplasmic reticulum Ca²⁺-ATPase (SERCA) pumps comprising:
identifying a subject having a reduced cardiac function caused by a defect in the function of the one or more SERCA pumps; and
providing the subject with an effective amount of an activator that enhances an activity of the one or more SERCA pumps.

32. The method of claim 31, wherein the reduced cardiac function is age-related, is oxidative stress related or is in the left ventricle.

33. (canceled)

34. (canceled)

35. The method of claim 31, wherein the activator is selected from CDN1163, ranolazine, istaroxime, or gingerol.

36. The method of claim 31, wherein the one or more SERCA pumps is selected from SERCA1, 2, 3 or any isoforms of SERCA1, 2 or 3.

37. A method of detecting and treating a subject with skeletal muscular atrophy caused by a defect in the function of sarco/endoplasmic reticulum Ca²⁺-ATPase (SERCA) pumps comprising:
obtaining a muscle biopsy from the subject;
detecting if the sample has decreased levels of SERCA pump activity; and
treating the subject with an effective amount a SERCA activator that enhances and/or restores anactivity of the one or more SERCA pumps.

38. A method of identifying a candidate agent for treating skeletal muscular atrophy caused by a defect in the function of one or more sarco/endoplasmic reticulum Ca²⁺-ATPase (SERCA) pumps, the method comprising:
(a) contacting a mammalian cell with a test agent;
(b) measuring an expression level and/or activity level of the one or more SERCA pumps in the mammalian cell relative to a reference value following the contacting;
(c) determining that the test agent caused an increase in the expression level and/or activity level relative to the reference value; and
(d) identifying the test agent as a candidate agent for treating skeletal muscular atrophy caused by a defect in the function of the one or more SERCA pumps.

39. A method of treating a subject in need of at least one of: muscle regeneration, reduced muscle necrosis, improved mitochondrial morphology, extended lifespan, protection from contraction-induced injuries, or protection from Ca′-driven necrosis in the gastrocnemius muscle, comprising providing the subject with an effective amount of an activator of one or more sarco/endoplasmic reticulum Ca²⁺-ATPase (SERCA) pumps sufficient to induce muscle regeneration, reduced muscle necrosis, improve mitochondrial morphology, extended lifespan, protect from contraction-induced injuries, or protect from Ca′-driven necrosis in the gastrocnemius muscle.

40. The method of claim 39, wherein the candidate agent is a derivative of CDN1163, ranolazine, istaroxime, or gingerol.
